(19) United States
(12) Patent Application Publication
(10) Pub. No.: US 2026/0331277 A1
(43) Pub. Date: Oct. 22, 2026

(54) ENHANCING ULTRASOUND DIAGNOSTICS SYSTEM USING EXTERNAL APPLICATION

(71) Applicant: GE Precision Healthcare LLC, Waukesha, WI (US)

(72) Inventors: Michael Washburn, Brookfield, WI (US); Kazuaki Okamoto, Tokyo (JP); Anurag Velekkatt Sunil Kumar, Milwaukee, WI (US)

(73) Assignee: GE Precision Healthcare LLC, Waukesha, WI (US)

(21) Appl. No.: 19/182,483

(22) Filed: Apr. 17, 2025

(51) Int. Cl. G16H 40/67; H04L 65/60; H04L 67/10; H04L 67/12

(52) U.S. Cl. CPC G16H 40/67; H04L 65/60; H04L 67/10; H04L 67/12

(57) ABSTRACT

Systems and methods for enhancing ultrasound imaging systems using external applications are described herein. In one example, an ultrasound imaging system includes a transducer, a matching layer, a damping block, a display screen, and a processing circuit. The processing circuit includes a processor coupled to a memory device storing instructions thereon that, when executed, cause the processing circuit to perform operations. The operations include obtaining ultrasound image data from the transducer, transmitting the ultrasound image data over a network to a remote computing system, receiving display data over the network from the remote computing system, and displaying the display data on the display screen. The ultrasound data is transmitted to the remote computing system via a first data transmission protocol, and the display data is received from the remote computing system via a second data transmission protocol.

### FIELD

Embodiments of the subject matter disclosed herein relate to ultrasound imaging, and more particularly, to the use of external applications configured to enhance the diagnostic capabilities of an ultrasound imaging system.

### BACKGROUND

During a medical imaging scan, a plurality of medical images of a patient are obtained by a technician, such as a sonographer, to measure or detect various aspects of anatomical features present within the medical images. Furthermore, external applications (e.g., off-system computing devices) may be used to process the medical images and provide various capabilities such as enhanced diagnostic tools.

### SUMMARY

An embodiment relates to an ultrasound imaging system. The ultrasound imaging system includes a transducer configured to transmit and receive an ultrasound signal. The ultrasound imaging system includes a matching layer configured to have an acoustic impedance match between a tissue to be imaged and a material of the transducer. The ultrasound imaging system includes a damping block configured to absorb ultrasound energy. The ultrasound imaging system includes a display screen. The ultrasound imaging system includes a processing circuit including a processor coupled to a memory device storing instructions thereon that, when executed, cause the processing circuit to perform operations. The operations include obtaining ultrasound image data from the transducer. The operations include transmitting, via a first data transmission protocol, the ultrasound image data over a network to a remote computing system. The operations include receiving, via a second data transmission protocol, display data over the network from the remote computing system. The operations include displaying the display data on the display screen.

Another embodiment relates to a medical imaging system. The medical imaging system includes a processing circuit having a processor coupled to a memory device storing instructions thereon that, when executed, cause the processing circuit to perform operations. The operations include obtaining medical image data. The operations include transmitting, via a first data transmission protocol, the medical image data over a network to a remote computing system. The operations include receiving, via a second data transmission protocol, display data over the network from the remote computing system. The operations include displaying the display data on a display screen of the medical imaging system.

Another embodiment relates to a method. The method includes obtaining, by a processing circuit of an ultrasound imaging system, ultrasound image data from a transducer of the ultrasound imaging system. The method includes transmitting, by the processing circuit and via a first data transmission protocol, the ultrasound image data over a network to a remote computing system. The method includes receiving, by the processing circuit and via a second data transmission protocol, display data over the network from the remote computing system. The method includes displaying, by the processing circuit, the display data on a display screen of the ultrasound imaging system.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a schematic diagram of a computing environment used to perform an ultrasound imaging procedure, according to an example embodiment.

FIG. 2 is an illustration of an ultrasound imaging system, according to an example embodiment.

FIG. 3 is a block diagram of the ultrasound imaging system of FIG. 2, according to an example embodiment.

FIG. 4 is a block diagram of a processing circuit of the ultrasound imaging system of FIG. 3, according to an example embodiment.

FIG. 5 is a flow chart illustrating a method for performing an ultrasound imaging procedure, according to an example embodiment.

FIG. 6 is a block diagram of a remote computing system used in the computing environment of FIG. 1, according to an example embodiment.

FIG. 7 is a flow chart illustrating a method for processing ultrasound image data using the remote computing system of FIG. 6, according to an example embodiment.

FIG. 8 is a diagram illustrating the computing environment of FIG. 1 being used to perform an ultrasound imaging procedure, according to an example embodiment.

FIG. 9 is a flow chart illustrating a method for performing an ultrasound imaging procedure using the computing environment of FIG. 1, according to an example embodiment.

FIG. 10 is a flow chart illustrating another method for performing an ultrasound imaging procedure using the computing environment of FIG. 1, according to an example embodiment.

### DETAILED DESCRIPTION

Referring generally to the figures, systems and methods for enhancing an ultrasound diagnostics system with an external solution application are disclosed. More specifically, the systems and methods described herein include transmitting ultrasound image data over a network to a remote computing system, receiving display data over the network from the remote computing system, and displaying the display data on a display of the ultrasound diagnostics system.

In existing medical imaging devices, remote (e.g., off-system) computing devices may be used to enhance diagnostic capabilities. More specifically, the remote computing devices receive data from the medical imaging device, process the data, and provide various diagnostic capabilities in response. Such systems, however, use an external software (e.g., cloud-based, application programming interface (API)-based, etc.) to process the data and send back the result, which is then directly shown on a display of the medical imaging device. Accordingly, existing medical imaging devices are continuously updating and replacing their software to adapt to the different external software used by the remote computing devices. This consistent modification of internal software for the purpose of compatibility with external software leads to extra development costs of medical devices and medical device registration issues.

Furthermore, existing medical imaging devices may implement a remote desktop connection or other remote display technique. In such systems, a remote computer processes and analyzes medical image data. The medical image data is captured by a medical imaging device (e.g., an ultrasound imaging system, etc.), and is converted into the digital imaging and communications in medicine (DICOM) format for processing by the remote computer. Then, the results of the analysis performed by the remote computer are shown on a screen of the medical imaging device. In such systems, the remote desktop connection or other remote display technique remotely displays the remote computer’s screen on the medical imaging device.

The systems and methods described herein, however, provide a technical solution to existing systems by using a remote desktop connection to access any third-party software through a medical imaging device interface. In this way, the medical imaging device does not have to change its software before using different external applications. Once the remote desktop connection is installed on the medical imaging device, any third-party software can be accessed without software modifications and re-registration of the medical imaging device. For example, using the systems and methods described herein, an ultrasound diagnostics system does not have to upgrade its own software in order to use enhanced diagnostic techniques performed by a third-party application. In this way, a user of the ultrasound diagnostics system is able to use enhanced diagnostics-support capabilities directly via the ultrasound diagnostics system.

The implementations described herein address a technical problem by providing enhanced data integration and analysis capabilities, which deliver a particular technical solution that streamlines and refines generation and transmittal of medical images. More specifically, the systems and methods described herein provide for a remote desktop connection between a medical imaging device and a remote computing system configured to minimize the work of modifying the medical imaging device software in order to adapt to various third-party software of remote computing systems. That is, once the remote desktop interface is integrated into the medical imaging device, a variety of third-party software can be integrated into the medical imaging device display such that the third-party software is shown and operated via the display. Accordingly, this approach provides a specific technical improvement to various technical problems, including those set forth herein.

The systems described herein may also reduce processing power by performing various processing operations simultaneously, rather than performing a plurality of processing operations individually and consuming unnecessary processing power. That is, the systems and methods described herein result in more efficient integration of external applications (e.g., remote computing systems, third-party software, etc.) within medical imaging devices. For example, with the remote desktop connection and display techniques described herein, the remote computing system directly receives binary ultrasound image data frame-by-frame in real-time from the ultrasound diagnostics system. Then, the remote computing system processes and analyzes the binary ultrasound data in real-time. In this way, the ultrasound data is not first converted or translated into a standard format (e.g., DICOM) before being received by the remote computing system. The remote computing system displays the results of the data analysis in real-time, and the ultrasound diagnostics system displays the remote computing system’s screen using the remote desktop connection and display techniques in real-time.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

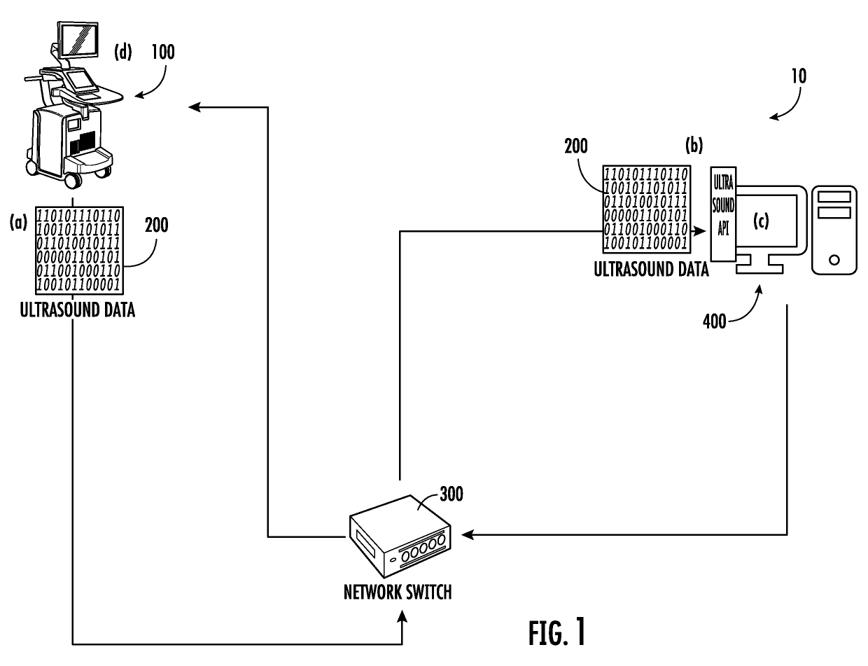

Referring to FIG. 1, a computing environment 10 used to perform an ultrasound imaging procedure is shown. As shown, the computing environment 10 includes an ultrasound imaging system 100, a network switch 300, and a remote computing system 400. Furthermore, the computing environment 10 is configured to facilitate transmitting ultrasound data 200 from the ultrasound imaging system 100 to the remote computing system 400 via the network switch 300.

The ultrasound imaging system 100 may be used in a medical environment (e.g., hospitals, clinics, etc.), for example, by a sonographer, technician, or other clinician certified to collect ultrasound data from a patient. Although the systems and methods are described herein in the context of the ultrasound imaging system 100, it should be appreciated that the remote desktop connection and display techniques described herein may be implemented using any of a variety of medical imaging systems (e.g., medical resonance imaging, x-ray, computed tomography, positron emission tomography, etc.).

As described in greater detail herein, the ultrasound imaging system 100 is configured to obtain and transmit the ultrasound data 200. The ultrasound data 200 is transmitted to the remote computing system 400 in real-time relative to when the ultrasound data 200 is obtained by the ultrasound imaging system 100. In some embodiments, the ultrasound image data 200 refers to binary data. Binary ultrasound data refers to the digital representation of ultrasound signals using the binary number system, which includes digits 0 and 1. When the ultrasound imaging system 100 captures echoes from tissues and organs, as described below, these echoes are converted into electrical signals. These signals are then digitized into binary form, allowing for efficient storage, processing, and analysis by computers (e.g., the ultrasound imaging system 100, the remote computing system 400, etc.). This binary data can be manipulated to enhance image quality, perform measurements, and apply various diagnostic algorithms. The use of binary data ensures that ultrasound images are precise and can be easily shared and interpreted across different digital platforms, such as between the ultrasound imaging system 100 and a plurality of remote computing systems (e.g., remote computing system 400).

As shown in FIG. 1, the ultrasound data 200 is transmitted from the ultrasound imaging system 100 to the remote computing system 400 via the network switch 300. The network switch 300 configured to connect multiple systems or devices (e.g., ultrasound imaging system 100, remote computing system 400, etc.) within a network and facilitate communication between them. Further, the network switch 300 directs data only to the specific device it is intended for, improving network efficiency and security. For example, if the ultrasound data 200 is being transmitted to an external application configured to perform cardiovascular diagnostics, the network switch 300 may be configured to direct the ultrasound data 200 to a remote computing system 400 associated with the cardiovascular diagnostics application, and not to a remote computing system 400 associated with a fetal measurements application.

As shown, the remote computing system 400 receives the ultrasound data 200 from the ultrasound imaging system 100 via the network switch 300. More specifically, the remote computing system 400 is shown to receive the ultrasound data 200 via an ultrasound API. The ultrasound API facilitates integration of ultrasound imaging functionalities into the remote computing system 400. In other words, the ultrasound API provides tools and protocols for accessing and controlling the ultrasound imaging system 100, which allows for real-time image streaming, parameter adjustments, data retrieval, and so on. For instance, the remote computing system 400 can use the ultrasound API to receive and display the ultrasound data 200 in real-time, adjust imaging settings (e.g., depth, gain, etc.), and analyze the ultrasound data 200 for research or diagnostic purposes. This integration facilitates the development of innovative medical applications, enhances diagnostic capabilities, and supports advanced research in medical imaging and artificial intelligence.

Although FIG. 1 is shown to include a first remote computing system 400, it should be appreciated that, in some embodiments, the computing environment 10 may include a plurality of remote computing systems 400. That is, each of the remote computing systems 400 may be configured to execute an external solution application used by the ultrasound imaging system 100 during an ultrasound imaging procedure. In some embodiments, the external application may relate to image processing, pathology detection, artificial intelligence capabilities, and so on. Additionally or alternatively, the external application may relate to a specific application for which the ultrasound imaging system 100 is being used (e.g., an echocardiogram, a fetal examination, a lung scan, etc.).

The remote computing system 400 converts the ultrasound data 200 such that the ultrasound data 200 is displayed on a screen of the remote computing system 400, and then the same display of the ultrasound data 200 is provided to the ultrasound imaging system 100 via the network switch 300. In other words, the ultrasound imaging system 100 projects the screen of the remote computing system 400 on its own display (e.g., display device 132) via the network switch 300. In this way, an operator of the ultrasound imaging system 100 (e.g., a sonographer) can view the ultrasound data 200 in real-time and can operate the external application (e.g., the remote computing system 400) via the ultrasound imaging system 100.

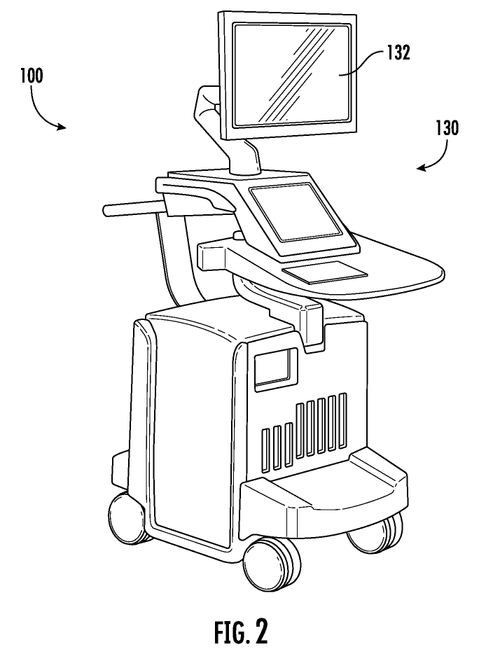

Referring to FIG. 2, the ultrasound imaging system 100 is shown in greater detail. Specifically, the ultrasound imaging system 100 is shown to include a user interface 130 and a display device 132.

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

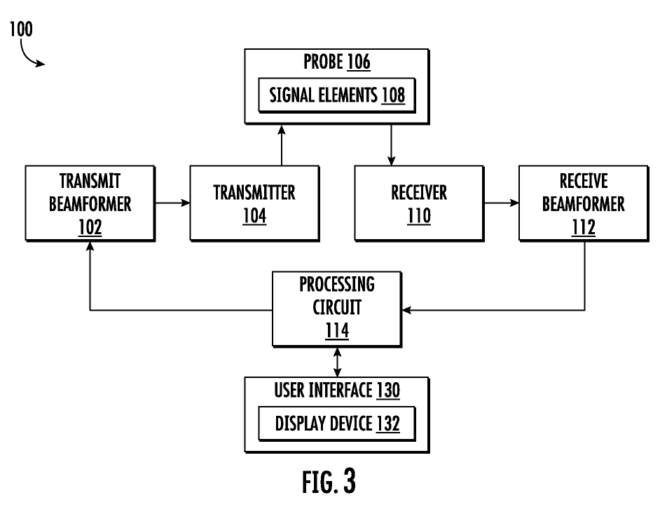

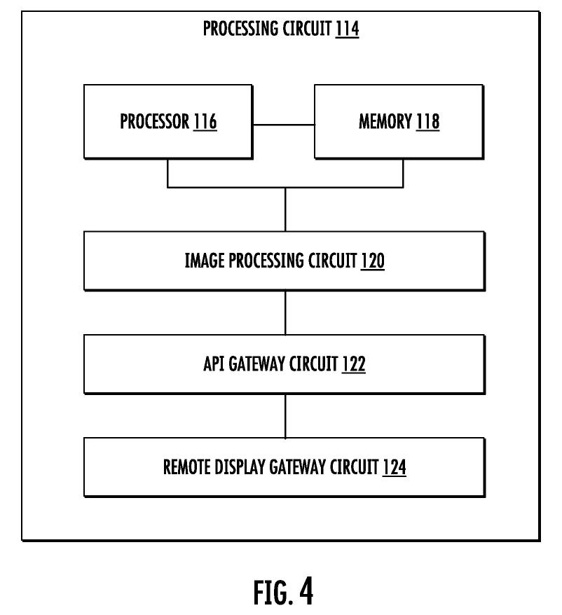

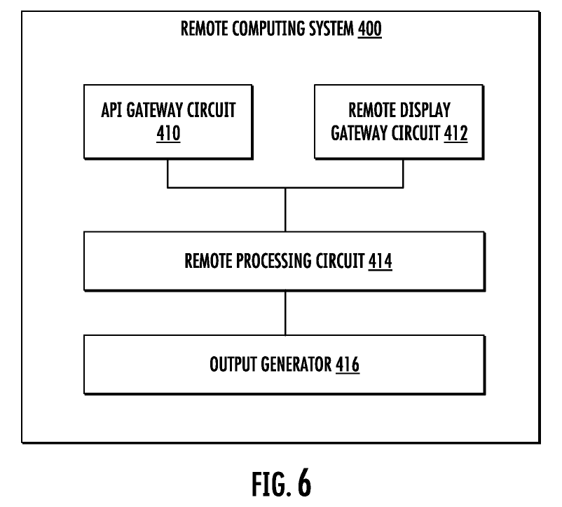

According to some embodiments, the user interface 130 may be physically separate from the rest of the ultrasound imaging system 100 (e.g., the transmit beamformer 102, the transmitter 104, the probe 106, the receiver 110, the receive beamformer 112, and/or the processing circuit 114, as shown in FIG. 3). The user interface 130 may communicate with a processor (e.g., processor 116 as shown in FIG. 4, remote processing circuit 414 as shown in FIG. 6) through a wireless protocol, such as Wi-Fi, Bluetooth, wireless local area network (WLAN), near-field communication, and so on. According to some embodiments, the user interface 130 may communicate with the processor through an application programming interface (API) (e.g., the ultrasound API associated with the remote computing system 400). In some embodiments, the user interface 130 may include physical controls such as one or more of buttons, sliders, a rotary knob, a mouse, a keyboard, a trackball, hard keys linked to specific actions, soft keys that may be configured to control different functions, and so on.

As shown in FIG. 2, the ultrasound imaging system 100 may also include a display device 132. In some embodiments, as shown in FIG. 3, the display device 132 may be part of the user interface 130. The display device 132 may be a screen configured to display a graphical user interface (GUI) based on an instruction from a memory (e.g., memory 118, as shown in FIG. 4). The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132.

The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring to FIG. 3, a block diagram of the ultrasound imaging system 100 is shown. As shown in FIG. 3, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a fetal skull at a first location of the probe 106. Then, the position sensor may track the movement of the probe 106 relative to the fetal skull in order to identify successive locations of the probe 106. In some embodiments, the position sensor may transmit position data to be stored within the ultrasound imaging system 100 (e.g., in memory 118, as shown in FIG. 4).

The probe 106 may include a transducer configured to transmit and receive an ultrasound signal. In some embodiments, as shown in FIG. 3, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during a fetal examination, a sonographer or other clinician may navigate the probe 106 proximate to a patient’s uterus so that the signal elements 108 in the probe 106 emit the pulsed ultrasonic signals into the patient’s uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance match between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as shown in FIG. 4) may be configured to perform some or all of the functions associated with the receive beamformer 112.

Although the transmit beamformer 102, the transmitter 104, the receiver 110, and the receive beamformer 112 are shown in FIG. 3 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 3, the ultrasound imaging system 100 is shown to include a processing circuit 114. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The ultrasound imaging system 100 is also shown to include the user interface 130 and the display device 132, as described above in greater detail above with reference to FIG. 2.

Referring to FIG. 4, the processing circuit 114 of the ultrasound imaging system 100 is shown in greater detail. As shown, the processing circuit 114 may include at least one processor 116, a memory 118, an image processing circuit 120, an API gateway circuit 122, and a remote display gateway circuit 124. In this way, the processing circuit 114 may be structured or configured to execute or implement the instructions, commands, and/or control processes described herein with respect to the processor 116, the memory 118, the image processing circuit 120, the API gateway circuit 122, and the remote display gateway circuit 124.

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132.

As shown in FIG. 4, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes’ worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120. The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a fetal ultrasound and may therefore include various images of a patient’s uterus and the fetal anatomy contained therein. The image processing circuit 120 may include multiple deep learning-based models configured to analyze the image data. For example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

The processing circuit 114 also includes the API gateway circuit 122 and the remote display gateway circuit 124. Both the API gateway circuit 122 and the remote display gateway circuit 124 are configured to facilitate integration of external applications (e.g., associated with remote computing systems 400) within the ultrasound imaging system 100, as described herein. For example, the API gateway circuit 122 may be configured to facilitate transmitting the ultrasound data 200 from the ultrasound imaging system 100 to the remote computing system 400, and the remote display gateway circuit 124 may be configured to facilitate transmitting display data (e.g., display data 250, as described below) from the remote computing system 400 to the ultrasound imaging system 100.

The API gateway circuit 122 refers to an intermediary between the ultrasound imaging system 100 and the remote computing system 400. The API gateway circuit 122 facilitates secure and efficient communication between the ultrasound imaging system 100 and the remote computing system 400 by facilitating authentication, authorization, request routing, and data transformation. Furthermore, the API gateway circuit 122 provides the ultrasound imaging system 100 with access to additional functionality, such as image acquisition and processing, through standardized API calls. Using the API gateway circuit 122, the ultrasound imaging system 100 can support various external applications, including remote diagnostics, telemedicine, advanced data analysis, and so on.

The remote display gateway circuit 124 facilitates the transmission of ultrasound images and data (e.g., display data 250) to remote displays, enabling real-time monitoring and diagnostics from different locations. For example, the remote display gateway circuit 124 may allow a user of the ultrasound imaging system 100 to interact with a display of data being processed at the remote computing system 400. In other words, the data may be viewed and interacted with at the ultrasound imaging system 100 while being processed at the remote computing system 400 in real-time. The remote display gateway circuit 124 communicates with remote devices (e.g., the ultrasound imaging system 100, the remote computing system 400, etc.) via a network (e.g., using the network switch 300). As described herein, such communication allows users (e.g., sonographers, clinicians, etc.) to view and interact with ultrasound images (e.g., display data 250) on the display device 132.

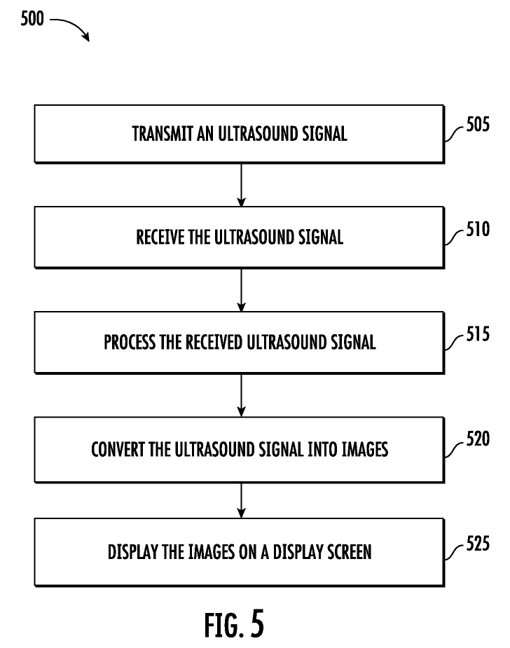

Referring to FIG. 5, a flow chart is shown illustrating a method 500 for performing an ultrasound imaging procedure using an ultrasound imaging system. In at least one embodiment, the ultrasound imaging system referred to by method 500 is the ultrasound imaging system 100 described above with reference to FIGS. 1-4, and method 500 may be implemented by the ultrasound imaging system 100. In some embodiments, method 500 may be implemented as executable instructions in a memory of the ultrasound imaging system 100, such as the memory 118 of FIG. 4. Additionally or alternatively, one or more steps of method 500 may be implemented by the remote computing system 400.

At step 505, method 500 may include transmitting an ultrasound signal. The signal elements 108 (e.g., the transducer) of the probe 106 may transmit the ultrasound signal at step 505 by emitting high-frequency sound waves. For instance, when the transducer is brought into contact with a patient’s body, the transducer sends the sound waves into the body. As the sound waves travel through different tissues and organs, they encounter boundaries between different types of tissues, such as muscle and bone. At these boundaries, some of the sound waves are reflected back to the transducer, while others continue to travel deeper into the body.

Then, at step 510, the transducer captures the sound waves that are reflected back from the tissue boundaries. These reflected waves, or echoes, are converted into electrical signals by the transducer. The strength and timing of these echoes provide information regarding the depth and density of the tissues they encountered at step 505 (which is used to create accurate images of the internal structures).

At step 515, the electrical signals are processed by ultrasound imaging system 100. In other words, once the ultrasound signals are received by the transducer and converted into electrical signals, the electrical signals undergo processing to enhance their quality and extract meaningful information. More specifically, the ultrasound imaging system 100 (e.g., the image processing circuit 120) may be configured to amplify the electrical signals and filter out noise. Advanced algorithms may be applied to analyze the echoes, determining the origin and intensity of the echoes. In some embodiments, step 515 may include adjusting parameters such as gain and depth to optimize the clarity of resulting images (e.g., displayed at step 525, as described below).

At step 520, the processed ultrasound signals are converted into ultrasound images. The ultrasound imaging system 100 is configured to translate the electrical signals into a visual format that represents the internal structures of the body. Specifically, step 520 may include mapping the intensity and location of the echoes to corresponding points on the ultrasound image.

The ultrasound images are displayed on a display screen (e.g., display device 132) at step 525 for real-time viewing. The ultrasound images may be displayed in grayscale, with varying shades representing different tissue densities. The ultrasound images can be adjusted for brightness, contrast, and other parameters to enhance visibility.

Referring to FIG. 6, a block diagram of the remote computing system 400 is shown. As shown in FIG. 6, the remote computing system 400 may include an API gateway circuit 410, a remote display gateway circuit 412, a remote processing circuit 414, and an output generator 416.

As described above with reference to FIG. 4, the API gateway circuit 410 and the remote display gateway circuit 412 are configured to facilitate integration of external applications (e.g., associated with the remote computing system 400) within the ultrasound imaging system 100. For example, the API gateway circuit 410 may be configured to facilitate receiving the ultrasound data 200 from the ultrasound imaging system 100, and the remote display gateway circuit 412 may be configured to facilitate transmitting display data (e.g., display data 250, as described below) to the ultrasound imaging system 100.

The remote processing circuit 414 refers to a processing circuit configured to perform the operations of the external application associated with the remote computing system 400. For example, the remote processing circuit 414 may be configured to perform operations on the ultrasound data 200 relating to image analysis, pathology detection, artificial intelligence capabilities, and so on, depending on the external application being used.

The output generator 416 is configured to generate a display of ultrasound data (e.g., display data 250) for projection onto a display device (e.g., display device 132) of the ultrasound imaging system 100. More specifically, the output generator 416 receives processed data from the remote processing circuit 414 and generates a visual display of the processed data to be transmitted to the ultrasound imaging system 100 via the network switch 300. In this way, a user of the ultrasound imaging system 100 can view real-time ultrasound images being processed at a remote location (e.g., the remote computing system 400). The output generator 416 generates the display data 250 such that the transmitted images maintain high quality and resolution, providing accurate and reliable visual information to the ultrasound imaging system 100 for medical assessment.

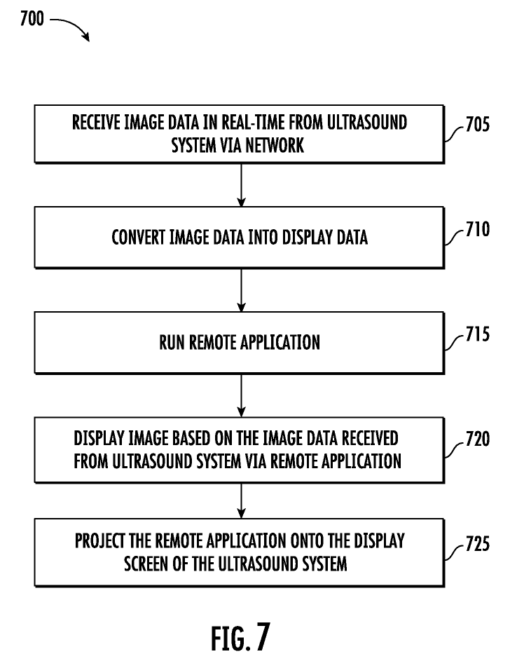

Referring to FIG. 7, a flow chart is shown illustrating a method 700 for processing ultrasound imaging data using a remote computing system. In at least one embodiment, the remote computing system referred to by method 700 is the remote computing system 400 described above with reference to FIGS. 1 and 6, and method 700 may be implemented by the remote computing system 400. In some embodiments, method 700 may be implemented as executable instructions in a memory of the remote computing system 400.

At step 705, image data is received in real-time from the ultrasound imaging system 100 via a network. In some embodiments, the image data received at step 705 refers to the ultrasound data 200, and is received via the network switch 300. More specifically, the API gateway circuit 410 of the remote computing system 400 may receive the image data at step 705 via an ultrasound-API, as described above with reference to FIG. 1.

The image data received at step 705 is converted into display data at step 710. That is, the ultrasound data 200 (e.g., the binary data) may be converted into visual data to be displayed on the remote computing system 400 and, in turn, on the ultrasound imaging system 100 (e.g., on the display device 132). In other words, the remote processing circuit 414 converts the ultrasound data 200 into visual image data such that the data displayed on the screen of the remote computing system 400 can be projected onto the display device 132 of the ultrasound imaging system 100.

Step 715 includes running the remote application. That is, running the remote application at step 715 refers to the remote processing circuit 414 performing the operations associated with the external application associated with the remote computing system 400. That is, the operations may include image processing, pathology detection, artificial intelligence capabilities, etc., depending on the external application being used by the ultrasound imaging system 100.

At step 720, an image is displayed based on the image data received at step 705 from the ultrasound imaging system 100 via the remote computing system 400. That is, the image is displayed on the remote computing system 400 configured to visually represent the binary ultrasound data 200 received from the ultrasound imaging system 100.

At step 725, the remote application is projected onto a display screen (e.g., display device 132) of the ultrasound imaging system 100. By projecting the remote application, the image displayed on the remote computing system 400 at step 720 is projected on the display screen of the ultrasound imaging system 100, as well as any successive processing performed by the remote application. For instance, if the user of the ultrasound imaging system 100 interacts with the display via the display device 132, the remote application is configured to receive such interaction and may perform successive processing of the ultrasound data 200 in response. In this instance, the display projected on the display screen is updated in real-time based on the operations of the remote application while it is running.

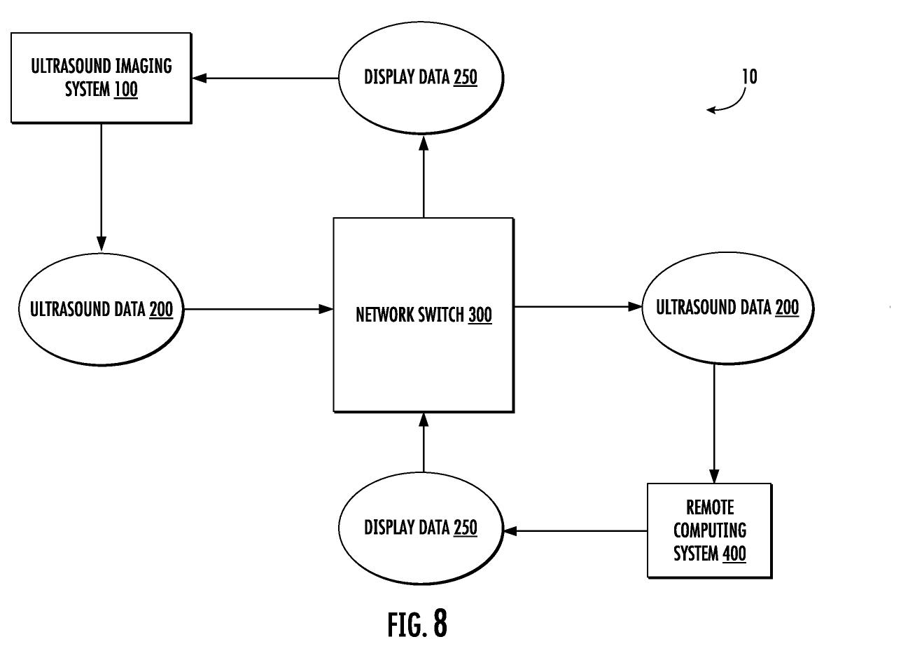

Referring to FIG. 8, a diagram illustrating the computing environment 10 being used to perform an ultrasound imaging procedure is shown. As shown in FIG. 8, the ultrasound imaging system 100 transmits the ultrasound data 200 to the remote computing system 400 via the network switch 300. The ultrasound data 200 is transmitted to the remote computing system 400 in real-time relative to when it is received by the transducer (e.g., at step 510 of method 500). Furthermore, the ultrasound data 200 maybe transmitted via a first data transmission protocol, the first data transmission protocol using an API (e.g., the ultrasound data 200 is transmitted using the API gateway circuit 122, the ultrasound data 200 is received by the API gateway circuit 410). Once the ultrasound data 200 is received, the remote computing system 400 converts the ultrasound data 200 (which is binary data, as described above with reference to FIG. 1) to display data 250.

Then, the ultrasound imaging system 100 receives the display data 250 from the remote computing system 400 via the network switch 300. The display data 250 may be received via a second data transmission protocol that is different from the first data transmission protocol (e.g., using the API), the second data transmission protocol being a remote display protocol (e.g., the display data 250 is transmitted by the remote display gateway circuit 412, the display data 250 is received by the remote display gateway circuit 124). As described above, the display data 250 is displayed on the display device 132 in real-time with respect to when the ultrasound image data is obtained from the transducer.

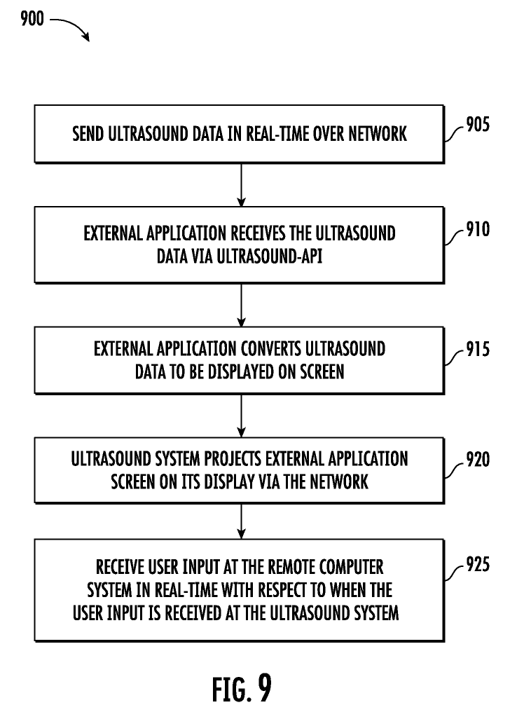

Referring to FIG. 9, a flow chart is shown illustrating a method 900 for performing an ultrasound imaging procedure using the computing environment 10. In at least one embodiment, method 1000 may be implemented by the ultrasound imaging system 100, the network switch 300, and the remote computing system 400. In some embodiments, method 900 may be implemented as executable instructions in a memory of the ultrasound imaging system 100 (such as memory 118), the network switch 300, or the remote computing system 400.

At step 905, ultrasound data (e.g., ultrasound data 200) is sent in real-time over a network (e.g., network switch 300). The ultrasound data may be obtained by the transducer of the ultrasound imaging system 100 and transmitted to the remote computing system 400. More specifically, the ultrasound data may be transmitted using a first communication protocol (e.g., an API communication protocol).

At step 910, an external application (e.g., remote computing system 400) receives the ultrasound data via an ultrasound-API. For example, the ultrasound data may be received by the API gateway circuit 410 of the remote computing system 400. The external application may be a remote computing system used by the ultrasound imaging system 100 and configured to perform enhanced diagnostic or other image processing capabilities. For example, the external application may be configured to perform image processing, pathology detection, artificial intelligence capabilities, and so on.

Once the ultrasound data is received at step 910, the external application converts the ultrasound data to be displayed on its screen at step 915. For instance, the external application may convert the ultrasound data into display data 250 such that what is displayed on the external application’s screen is also what is displayed on the display device 132 of the ultrasound imaging system 100. At step 920, the ultrasound system projects the external application screen on its display via the network.

Then, at step 925, user input is received at the remote computing system 400 in real-time with respect to when the user input is received at the ultrasound imaging system 100. That is, an operator of the ultrasound imaging system 100 may interact with the projection of the external application via the display device 132, and the interaction may be recognized by the external application in real-time. In this way, the operator is able to see ultrasound images in real-time and operate the external application via the display device 132. In some embodiments, the user input may be transmitted by the remote display gateway circuit 124 to the remote display gateway circuit 412 via the network switch 300.

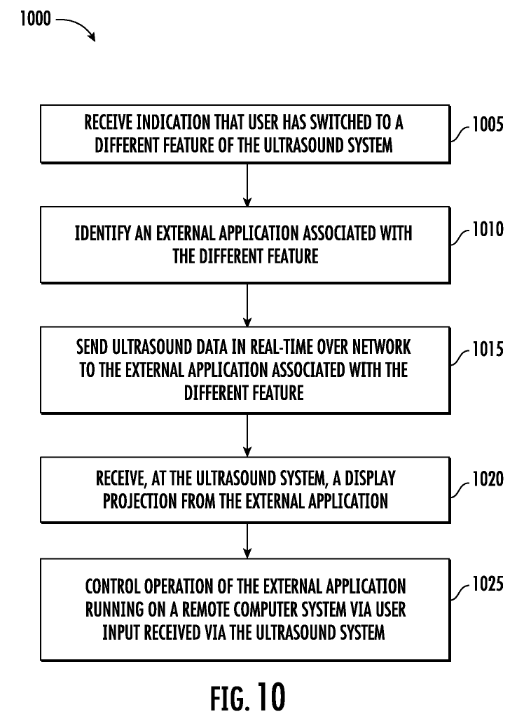

Referring to FIG. 10, a flow chart is shown illustrating a method 1000 for performing an ultrasound imaging procedure using the computing environment 10. In at least one embodiment, method 1000 may be implemented by the ultrasound imaging system 100, the network switch 300, and the remote computing system 400. In some embodiments, method 1000 may be implemented as executable instructions in a memory of the ultrasound imaging system 100 (such as memory 118), the network switch 300, or the remote computing system 400.

At step 1005, method 1000 is shown to include receiving an indication that a user has switched to a different feature of the ultrasound imaging system 100. That is, the ultrasound imaging system 100 may be configured to implement a plurality of features operable by a plurality of available external applications (e.g., a plurality of remote computing systems 400). For example, an operator may use a feature configured to take fetal measurements. When this feature is being used, the ultrasound imaging system 100 may be used according to a first application of the plurality of available external applications (e.g., the first application configured to take fetal measurements). The first application may be compatible with the ultrasound imaging system 100 using a first remote computing system 400. Then, the operator may switch to using a different feature (e.g., echocardiogram) of the ultrasound imaging system 100.

At step 1010, an external application associated with the different feature is identified. That is, continuing with the example above, when an operator uses the echocardiogram feature of the ultrasound imaging system 100, the ultrasound imaging system 100 may be used according to a second application of the plurality of available external applications (e.g., the second application configured to facilitate an echocardiogram). Therefore, in response to the operator switching to the echocardiogram feature, the second application may be identified from among the plurality of available applications that are compatible with the ultrasound imaging system 100.

At step 1015, ultrasound data (e.g., ultrasound data 200) is sent in real-time over a network (e.g., the network switch 300) to the external application associated with the different feature (e.g., as identified at step 1010). For example, continuing with the example above, the ultrasound data may be sent to a second remote computing system 400 configured to implement the second application (e.g., for performing echocardiograms) within the ultrasound imaging system 100. As described above, the second remote computing system 400 may receive the ultrasound data 200 via an ultrasound-API (e.g., the ultrasound data 200 is transmitted using the API gateway circuit 122, the ultrasound data 200 is received by the API gateway circuit 410). Then, as described above, the second remote computing system 400 converts the ultrasound data 200 into the display data 250.

At step 1020, the method 1000 includes receiving, at the ultrasound system, a display projection from the external application. That is, the display projection is received from the external application being run on the second remote computing system 400. In this way, method 1000 may include, at step 1025, controlling operation of the external application running on the second remote computing system 400 via user input received via the ultrasound imaging system 100. That is, as described above, the operator is able to view ultrasound data and operate the external application (e.g., configured to facilitate an echocardiogram) via the display device 132.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a transducer configured to transmit and receive an ultrasound signal;
a matching layer configured to have an acoustic impedance match between a tissue to be imaged and a material of the transducer;
a damping block configured to absorb ultrasound energy;
a display screen; and
a processing circuit comprising a processor coupled to a memory device storing instructions thereon that, when executed, cause the processing circuit to perform operations comprising:
obtaining ultrasound image data from the transducer;
transmitting, via a first data transmission protocol, the ultrasound image data over a network to a remote computing system;
receiving, via a second data transmission protocol, display data over the network from the remote computing system; and
displaying the display data on the display screen.

2. The ultrasound imaging system of claim 1, wherein the ultrasound image data is binary data.

3. The ultrasound imaging system of claim 1, wherein the first data transmission protocol uses an application programming interface.

4. The ultrasound imaging system of claim 1, wherein the second data transmission protocol is a remote display protocol.

5. The ultrasound imaging system of claim 1, wherein displaying the display data on the display screen occurs in real-time with respect to when the ultrasound image data is obtained from the transducer.

6. The ultrasound imaging system of claim 1, the operations further comprising transmitting a user input received via the display screen over the network to the remote computing system in real-time with respect to when the processing circuit receives the user input.

7. The ultrasound imaging system of claim 1, wherein the ultrasound imaging system is configured to run a first application of a plurality of available applications, wherein the plurality of available applications are hosted across one or more remote computing systems of a plurality of remote computing systems.

8. The ultrasound imaging system of claim 7, wherein the plurality of available applications relate to at least one of image processing, pathology detection, or artificial intelligence capabilities.

9. The ultrasound imaging system of claim 7, wherein the remote computing system is a first remote computing system of the plurality of remote computing systems and is configured to facilitate use of the first application, and wherein when use of the ultrasound imaging system switches to a second application of the plurality of available applications, the operations further comprise:
sending the ultrasound image data to a second remote computing system of the plurality of remote computing systems, the second remote computing system configured to facilitate use of the second application; and
receiving a display projection from the second remote computing system.

10. A medical imaging system comprising:
a processing circuit having a processor coupled to a memory device storing instructions thereon that, when executed, cause the processing circuit to perform operations comprising:
obtaining medical image data;
transmitting, via a first data transmission protocol, the medical image data over a network to a remote computing system;
receiving, via a second data transmission protocol, display data over the network from the remote computing system; and
displaying the display data on a display screen of the medical imaging system.

11. The medical imaging system of claim 10, wherein the medical imaging system is an ultrasound imaging system comprising:
a transducer configured to transmit and receive an ultrasound signal;
wherein the medical image data is obtained by the transducer.

12. The medical imaging system of claim 10, wherein the medical image data is binary data.

13. The medical imaging system of claim 10, wherein the first data transmission protocol uses an application programming interface, and wherein the second data transmission protocol is a remote display protocol.

14. The medical imaging system of claim 10, wherein displaying the display data on the display screen occurs in real-time with respect to when the medical image data is obtained.

15. The medical imaging system of claim 10, wherein the medical imaging system is compatible with a plurality of available applications using a plurality of remote computing systems, wherein the remote computing system is a first remote computing system of the plurality of remote computing systems and is configured to facilitate use of a first application of the plurality of available applications, and wherein when a user switches from the first application to a second application of the plurality of available applications, the operations further comprise:
sending the medical image data to a second remote computing system of the plurality of remote computing systems, the second remote computing system configured to facilitate use of the second application; and
receiving a display projection from the second remote computing system.

16. A method comprising:
obtaining, by a processing circuit of an ultrasound imaging system, ultrasound image data from a transducer of the ultrasound imaging system;
transmitting, by the processing circuit and via a first data transmission protocol, the ultrasound image data over a network to a remote computing system;
receiving, by the processing circuit and via a second data transmission protocol, display data over the network from the remote computing system; and
displaying, by the processing circuit, the display data on a display screen of the ultrasound imaging system.

17. The method of claim 16, wherein the first data transmission protocol uses an application programming interface, and wherein the second data transmission protocol is a remote display protocol.

18. The method of claim 16, wherein displaying the display data on the display screen occurs in real-time with respect to when the ultrasound image data is obtained from the transducer.

19. The method of claim 16, wherein the method further comprises transmitting a user input received via the display screen over the network to the remote computing system in real-time with respect to when the processing circuit receives the user input.

20. The method of claim 16, wherein the ultrasound imaging system is compatible with a plurality of available applications using a plurality of remote computing systems, wherein the remote computing system is a first remote computing system of the plurality of remote computing systems and is configured to facilitate use of a first application of the plurality of available applications, and wherein when a user switches from the first application to a second application of the plurality of available applications, the method further comprises:
sending the ultrasound image data to a second remote computing system of the plurality of remote computing systems, the second remote computing system configured to facilitate use of the second application; and
receiving a display projection from the second remote computing system.
